(19) United States
(12) Patent Application Publication
(10) Pub. No.: US 2024/0358366 A1
(43) Pub. Date: Oct. 31, 2024

(54) SUTURE METHODS

(71) Applicant: Xeropedix, Inc., Boston, MA (US)

(72) Inventors: Carl Schanbacher, Boston, MA (US); Christian Potter, Milford, MA (US)

(21) Appl. No.: 18/648,237

(22) Filed: Apr. 26, 2024

Related U.S. Application Data
(60) Provisional application No. 63/617,225, filed on Jan. 3, 2024.
(60) Provisional application No. 63/462,564, filed on Apr. 28, 2023.

(51) Int. Cl. A61B 17/06; A61B 17/00

(52) U.S. Cl. CPC A61B 17/06066; A61B 17/06166; A61B 2017/00004

(57) ABSTRACT

Systems and methods are provided for wound closure and suturing of skin using round bodies or taper point suture needles. Surprising results found that tapered or round bodies suture needles do not require unreasonable force to pass through skin, reduce bleeding and damage to the tissue, reduce risk of suture pull-through in skin, reduce finger sticks, and reduce the amount of transferred material in cases of finger sticks, thereby reducing the risk of disease transmission.

### CROSS REFERENCE TO RELATED APPLICATIONS

This application claims priority to and the benefit of U.S. Prov. App. Ser. No. 63/462,564, filed Apr. 28, 2023, and 63/617,225, filed Jan. 3, 2024, the content of each of which is incorporated herein by reference in its entirety.

### TECHNICAL FIELD

This application relates generally to systems, methods and apparatuses, for skin sutures using round-bodied or tapered needles.

### BACKGROUND

Crude sutures and suture needles were used in wound closure at least as early as ancient Egypt and while both have developed significantly since then and are essential to modern medicine, because of their ubiquitousness and relative simplicity, they are often taken for granted and are not subjected to rigorous study. Instead common conceptions or misconceptions among many medical professionals lead to unquestioning use of certain needle designs for certain tasks without actual scientific evidence to support the assumptions underlying their recommended uses. Every suture needle shares three anatomical features: the eye, the body, and the point. Each of these subunits has several different options regarding size and shape, resulting in many distinct needle varieties. The eye may be looped or swaged to attach the suture thread, the body can be curved to various degrees and vary in size and thickness, and the point can be one of several specifically engineered shapes. The exact configuration of suture needle subunits depends on the qualities of the tissue to be sutured, so, in practice, a surgeon should be cognizant of their needle selection and support their choices with scientific evidence.

Many needle characteristics and their effect on tissue are already well-documented. The needle-suture junction is well-studied, and it is agreed that swaged needles, which crimp the end of the needle around the suture material, are superior to eyed needles due to their convenience, reliability, and reduced trauma to tissue. Furthermore, efforts are being made to bring the needle-to-suture width ratio as close to 1 as possible, to maximize the degree to which the suture material will plug the hole left by the needle, reducing bleeding. Similarly, the theoretical basis of needle body curvature and needle size selection is well-understood, largely dependent on the thickness of the necessary suture material, maneuverability required for proper suturing technique, as well as the surgeon's preference.

An area that is less well studied is needle point geometry, which can mainly be divided into either a cutting-edge or a taper point. Curved cutting-edge needles have at least two opposing cutting edges aligned parallel to the needle's axis of rotation and can be further subdivided into two main categories: conventional and reverse cutting. The former has a third cutting edge along the interior arc of the needle, producing a triangular cross-section at the tip. However, because of this design, conventional cutting needles pose the risk of tissue tear out as the inside edge can easily cut up and toward the edges of the wound as the needle is rotated. Reverse cutting needles instead place the third cutting edge along the outside arc of the needle, keeping the triangular cross section but theoretically reducing the risk of tissue tear out. Due to this, reverse-cutting needles are generally favored to conventional cutting needles. The tips of both cutting-edge varieties extends to the maximum diameter of the needle, at which point the body becomes round.

On the contrary, taper point needles are round-bodied along their entirety, displaying a circular cross-section at the tip. Various sources state that tapered needles are appropriate to use in cases where tissue is delicate and friable, because they pierce and spread tissue apart, rather than slicing like cutting-edge needles. However, the commonly accepted thought among medical professionals is that taper point or round-bodied needles should not be used for dense tissue like skin because the extra force needed to penetrate the tissue causes extra trauma and can bend the needle. Accordingly, reverse cutting needles and not taper point needles are used in suturing skin and in other dense tissues.

### SUMMARY

Systems and methods of the present disclosure run counter to conventional wisdom with respect to needle point geometry, finding that taper point or round-bodied suture needles offer several benefits over cutting and reverse cutting needles when used in skin sutures and other areas traditionally associated with needles with axial cutting edges.

Results of several studies show that tapered needles cause less tissue damage than cutting needles as they pass through tissue. Specifically, the taper needles spread tissue apart as opposed to cutting the tissue. Historically cutting needles were thought to be more suitable for dense tissue such as skin for the specific reason that they cut the tissue where spreading dense tissue with a taper point needle would require too much force, at the risk of bending the needle when suturing or causing damage to the tissue while forcing the needle through. However, there is little to no data to support that conception and taper point needles are in fact well suited to dense tissue such as skin, especially when considering the numerous benefits. In part because needles with cutting edges cut the tissue they create tears or weak points in the tissue that can serve as the starting point for further tears. The present invention recognizes that these defects, caused by needles with cutting edges, reduce the tear through strength of the sutured tissue relative to taper point needle sutures. Without wishing to be bound to any specific theory, it is believed that the round defect formed by the taper point needle more uniformly distributes forces of the suture, thereby increasing resistance to suture ripping or tear through. This phenomenon was found in both skin and cartilage models and sutures using taper point needles additionally showed reduced leakage in cardiovascular models relative to cutting edge needles. Another new finding was that the use of larger caliber sutures in conjunction with a taper point needle may be preferrable in even particularly fragile tissues such as eyelid skin because the larger gauge suture, perhaps further distributing pulling forces, exhibits increased resistance to tear through over narrower gauge sutures.

Due to the reduced tissue damage, it is believed that taper point needle sutures require less healing time and result in reduced scarring and improved aesthetic outcomes. The latter considerations are particularly important in skin sutures where the resulting scarring or lack thereof may be especially conspicuous.

An additional consideration in favor of the use of taper point needles is their reduced instance of glove tears and needle sticks as well as reduced transfer of fluids and reduced disease transmission when compared to needles with axial cutting edges. The cutting edges of such needles are capable of cutting through gloves, thereby breaking the sterile barrier between the medical professional and the patient they are treating. Taper point needles do not have cutting edges and, therefore reduce this risk, requiring a direct piercing motion to penetrate a gloved hand. Additionally, if a needle stick does penetrate a gloved hand, the taper point needle, as discussed above, displaces the glove material in a similar manner to how it displaces tissue, leaving a more round and more uniform defect when compared to a needle point with an axial cutting edge. Accordingly, the glove material is more likely to remain intact around the taper point needle and, it is believed, can serve to scrape or remove material (e.g., biofluids) from the surface of the needle as it passes through the glove and before it may enter the hand of the medical professional. In the event of a needle stick, therefore, a reduced amount of material may be transferred from a patient to the medical provider which can result in a reduced risk of disease transmission.

Aspects of the invention can include methods of cutaneous suturing, comprising the steps of providing a suture needle comprising a distal end comprising a point operable to pierce the epidermis, a proximal end attached to a suture thread, and a tapered portion between the distal and proximal end having a substantially round cross-section, wherein the suture needle does not comprise an axial cutting edge between the distal and proximal end Methods can include piercing an epidermal layer of a patient with the point of the distal end to form an opening in the epidermal layer; passing the suture needle through the opening; and pulling the suture thread through the opening using the suture needle. In some embodiments, the suture needle can be used to pierce and suture cartilage.

In certain embodiments, the suture needle can have a largest cross-sectional diameter between about 0.10 and about 0.97 mm. The proximal end of the suture needle can be swaged to the suture thread. The suture thread can be attached to the proximal end of the suture needle by an eye in the proximal end of the suture needle. The suture needle can be substantially straight along its axial length. The suture needle can be curved along its axial length. The suture needle can be curved between about a ⅜ circle and about a ½ circle. In various embodiments, the suture needle can comprise a flattened or textured surface portion along its axial length between the proximal and distal ends, said flattened or textured surface portion adapted to provide grip for a suturing tool.

In some embodiments, methods of the invention can include gripping the suture needle with a suturing tool at the flattened or textured surface portion during the piercing step. The suture thread can be absorbable. The suture thread can comprise gut, polydioxanone, poliglecaprone, or polyglactin. In some embodiments, the suture thread can be non-absorbable. The suture thread can comprise polypropylene, nylon, silk, or polyester. The suture thread can only be attached to only a single suture needle.

The epidermal layer of the patient can be located on the patient's face. In certain embodiments, the epidermal layer of the patient can be located on the patient's eyelid and the suture thread can be a 5-0 size or larger. The epidermal layer of the patient can be located on the patient's abdomen. In some embodiments, the epidermal layer of the patient can be located on the patient's back.

In certain aspects, kits and/or systems of the invention can include a suture needle comprising a distal end comprising a point operable to pierce the epidermis; a proximal end attached to a suture thread; and a tapered portion between the distal and proximal end having a substantially round cross-section, wherein the suture needle does not comprise an axial cutting edge between the distal and proximal end. Systems and/or kits can comprise a non-absorbable suture thread comprising polypropylene, nylon, silk, or polyester. The non-absorbable suture thread can comprise polypropylene and the suture thread can be swaged to the suture needle. In certain embodiments, a suture thread comprising polypropylene can be swaged to a single suture needle as opposed to two needles as used in certain cardiovascular suturing.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 illustrates steps of an exemplary method of cutaneous suturing.

FIG. 2 shows a 40× magnified photograph of tissue involvement of reverse cutting (green) and tapered (red) needles at a depth of 210 microns below the epidermal surface. Areas are 0.37 mm2 (reverse cutting) and 0.14 mm2 (tapered).

FIG. 3 shows porcine aorta blood loss by needle geometry. A) is a bar plot with mean and standard error. Results from a paired t-test between needle groups at each depth are displayed. B) is a scatter plot with mean and standard error for each needle geometry. Statistical significance between needle diameters was determined by one-way ANOVA and pairwise t-tests with Holm corrections. Only p-values for statistically significant differences are displayed.

FIG. 4 shows area of piercing defects produced by 0.64 mm Taper and Reverse Cutting needles at various skin depths. A paired t-test was used to determine statistical significance between needle geometries at each depth.

FIG. 5 shows the average force required to tear through an artificial skin matrix at a bite distance of 5 mm. Significance between groups was determined using pairwise t-tests (* p<0.05, ** p<0.01,*** p<0.001).

FIG. 6 is an illustration of how tension is distributed through defects produced by different needle geometries.

FIG. 7 is a box and whisker plot of results for both needle geometries at three needle diameter sizes.

FIG. 8 shows a taper point needles impact on woven mesh. A cylindrical shape can be seen in which no mesh fibers have been cut, instead, the fabric has been pushed in the direction of the force applied. 127×102 mm (300×300 DPI).

FIG. 9 shows a reverse cutting needles impact on woven mesh. A triangular defect was made, and several transected fibers are visualized at the apex of the needle hole defect. 127×123 mm (300×300 DPI).

FIG. 10 shows a typical force-displacement or time-displacement curve generated by a tensiometer with a photo of the experimental setup. The flat part of the curve (maxima) represents the force at which the suture material leads to a complete tear through.

FIG. 11 shows mean tear through (catastrophic failure) force bar-plot from suture gauge and suture needle geometry retention testing with nylon and polypropylene sutures. Using nylon, three USP gauge sizes were tested (3-0, 4-0, 5-0). Using polypropylene, one gauge size (5-0) was tested in three different needle geometries: precision conventional cutting, precision reverse cutting, and taper point.

### DETAILED DESCRIPTION

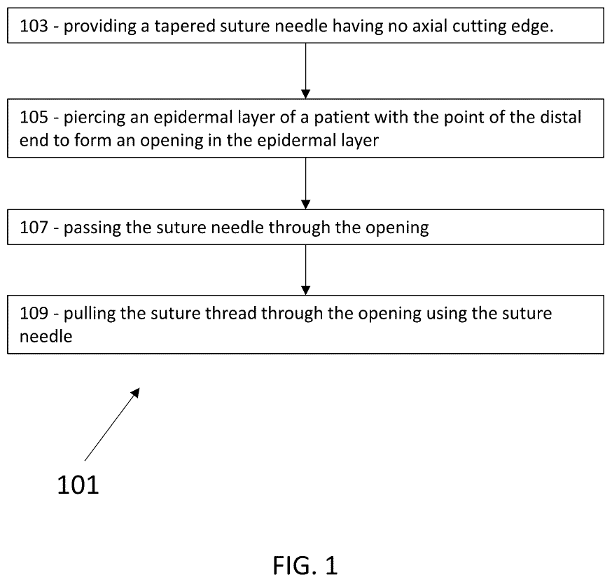

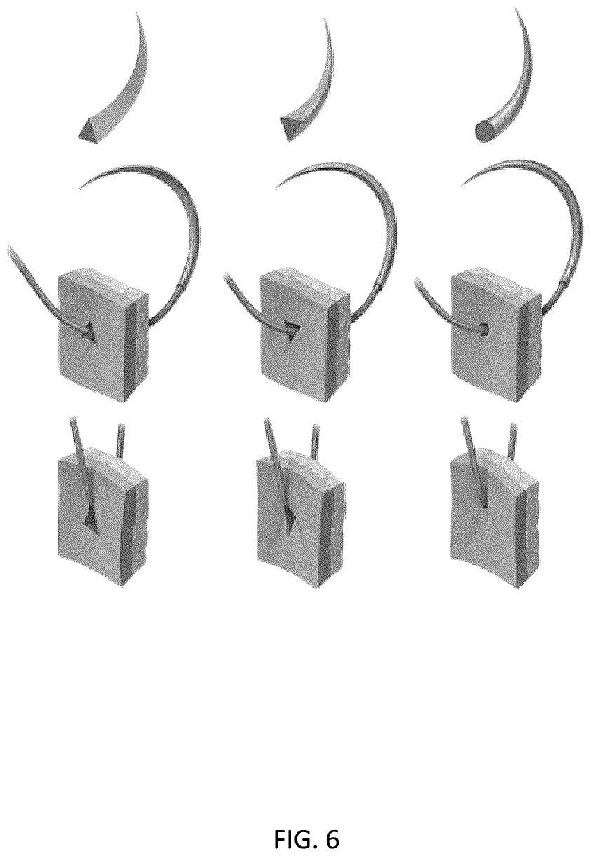

Systems and methods described herein relate to the nonconventional use of taper point or round bodied needles for sutures in skin and other dense tissues traditionally sutured using cutting or reverse cutting needles. FIG. 1 is a flow chart of a method 101 of cutaneous suturing. The described method can use a tapered suture needle having no axial cutting edge. Tapered needles have a round body that tapers from a wider body section to a central point at the end used to pierce tissue. The other end of the needle can be attached to a suture thread in order to pull the suture thread through the opening created in the tissue by the pointed end of the needle. While various point geometry variations exist including taper cut needles, as used herein, tapered or round-bodied needles refer to needles without a cutting edge as opposed to cutting or reverse cutting needle point geometries. Cutting, reverse cutting, and taper point needles are shown, from left to right, in FIG. 6. The taper point suture needles used in the systems and methods of the invention can be any size (length, or diameter) and curved as appropriate for their intended use and target tissue. In various embodiments, suitable taper point suture needles for the systems and methods described herein may be commercially available, such as those sold under needle codes RB-1, TF, SH, BB, C-1, or CT-1 by Ethicon (Bridgewater Township, NJ), or Sharpoint (Westwood, MA).

Methods 101 of cutaneous suturing can include providing 103 such a taper point needle and piercing 105 an epidermal layer of a patient with the distal (pointed) end of that needle to form an opening in the epidermal layer. The suture needle can then be passed 107 through the opening in order to pull 109 a suture thread attached to the proximal end of the suture needle through the opening following the needle. The steps of the method can be repeated on two proximate edges of tissue (e.g., in a laceration) to draw the two edges together (e.g., for wound closure).

It is believed that systems and methods of the invention, through the use of tapered needles, can reduce the damage to tissues through which the needles pass. As shown in the Examples below, taper point needles tend to spread tissue instead of cutting it. While this action is popularly believed to increase the amount of force needed to initially pierce and spread dense tissue such as skin relative to cutting or reverse cutting needles, the systems and methods herein recognize the surprising finding that the required force is not appreciably different and furthermore, does not result in increased tissue damage. Furthermore the benefits outweigh any minor increase in required force.

The benefits of taper point needles recognized herein include decreased tissue damage due to the spreading vs. cutting nature of the needle passage. This results in a round defect that better distributes the forces of the suture thread when pulling the tissue edges together. The cut edges resulting from cutting or reverse cutting needles, even when facing away from the tissue edge as in the case of reverse cutting needles, create focal points that tend to serve as the starting point for tissue tearing. Accordingly, the force required to a suture to tear through the skin is much lower when a needle with an axial cutting edge was used as opposed to a taper point needle. Beyond the surface opening, the cutting edges continue to pass through the full depth of the tissue to be sutured and the cutting edges can nick or cut microvasculature in the tissue, resulting in increased bleeding, tissue damage, and healing times.

Another advantage of taper point needles is that the round defect left by the needle conforms to the round suture better than a more triangular defect left by cutting or reverse cutting needles. Accordingly, the suture can better plug the hole to prevent leaking in cardiovascular sutures and generally better maintaining a barrier. This is especially complementary to the increased use of swaged needles with needle cross-sectional diameter configured to match the cross-sectional diameter of the suture thread.

In certain embodiments, the suture needle can have a largest cross-sectional diameter between about 0.10 and about 0.97 mm. The proximal end of the suture needle can be swaged to the suture thread. The suture thread can be attached to the proximal end of the suture needle by an eye in the proximal end of the suture needle. The suture needle can be substantially straight along its axial length. The suture needle can be curved along its axial length. The suture needle can be curved between about a ⅜ circle and about a ½ circle. In various embodiments, the suture needle can comprise a flattened or textured surface portion along its axial length between the proximal and distal ends, said flattened or textured surface portion adapted to provide grip for a suturing tool.

In some embodiments, methods of the invention can include gripping the suture needle with a suturing tool at the flattened or textured surface portion during the piercing step. The suture thread can be absorbable. The suture thread can comprise gut, polydioxanone, poliglecaprone, or polyglactin. In some embodiments, the suture thread can be non-absorbable. The suture thread can comprise polypropylene, nylon, silk, or polyester. The suture thread can only be attached to only a single suture needle.

Another new finding was that the use of larger caliber sutures in conjunction with a taper point needle can be preferable in even particularly fragile tissues such as eyelid skin because the larger gauge suture, perhaps further distributing pulling forces, exhibits increased resistance to tear through over narrower gauge sutures. Accordingly, the epidermal layer of the patient can be located on the patient's face. In certain embodiments, the epidermal layer of the patient can be located on the patient's eyelid and the suture thread can be a 5-0 size or larger, which can reduce the likelihood of a suture pulling through in the somewhat thin and fragile eyelid skin.

Historically, especially dense tissue such as the abdominal skin or back skin has been considered too difficult to push a taper point needle through. However, systems and methods of the invention recognize that the required increase in force over cutting-type needle point geometry is negligible and outweighed by the advantages disclosed herein.

In certain aspects, kits and/or systems of the invention can include a suture needle comprising a distal end comprising a point operable to pierce the epidermis; a proximal end attached to a suture thread; and a tapered portion between the distal and proximal end having a substantially round cross-section, wherein the suture needle does not comprise an axial cutting edge between the distal and proximal end. Systems and/or kits can comprise a non-absorbable suture thread comprising polypropylene, nylon, silk, or polyester. The non-absorbable suture thread can comprise polypropylene and the suture thread can be swaged to the suture needle. In certain embodiments, a suture thread comprising polypropylene can be swaged to a single suture needle as opposed to two needles as used in certain cardiovascular suturing.

Additional advantages of taper point needles include the observation that, due to the reduced tissue damage, taper point needle sutures require less healing time and result in reduced scarring and improved aesthetic outcomes. The latter considerations are particularly important in skin sutures where the resulting scarring or lack thereof can be especially conspicuous.

An additional consideration in favor of the use of taper point needles is their reduced instance of glove tears and needle sticks as well as reduced transfer of fluids and reduced disease transmission when compared to needles with axial cutting edges. The cutting edges of such needles are capable of cutting through gloves, thereby breaking the sterile barrier between the medical professional and the patient they are treating. Taper point needles do not have cutting edges and, therefore reduce this risk, requiring a direct piercing motion to penetrate a gloved hand. Additionally, if a needle stick does penetrate a gloved hand, the taper point needle, as discussed above, displaces the glove material in a similar manner to how it displaces tissue, leaving a more round and more uniform defect when compared to a needle point with an axial cutting edge. Accordingly, the glove material is more likely to remain intact around the taper point needle and, it is believed, can serve to scrape or remove material (e.g., biofluids) from the surface of the needle as it passes through the glove and before it can enter the hand of the medical professional. In the event of a needle stick, therefore, a reduced amount of material can be transferred from a patient to the medical provider which can result in a reduced risk of disease transmission. These observations and associated improvements in patient and medical provider safety are borne out in the following studies, the content of which are incorporated herein by reference in their entirety: Lefebvre, et al., 2008, An Enzyme-Mediated Assay to Quantify Inoculation Volume Delivered by Suture Needlestick Injury: Two Gloves Are Better Than One, J Am Coll Surg; 206:113-122, (In which multiple glove layers helped reduce contamination from needle sticks with cutting needles but did not offer any additional protection over a single layer for taper point needles.); and Sohn, et al., 2000, Detection of Surgical Glove Integrity, Am Surg. 2000 March; 66(3):302-6 (Finding that Injuries created with cutting and reverse-cutting solid needles were more likely to demonstrate a positive water load test than similar sized taper needles.).

Additionally, cutting and reverse cutting needles are composed of nickel and chromium and undergo a meticulous milling process to achieve a triangular blade shape. Upon examination with scanning electron microscopy, evidence of metal fragment shearing and shedding was observed in these surgical needles. This phenomenon occurred after passing the needle through human integument 30 times. Conversely, similar testing conducted on tapered or round-bodied needles did not reveal such shedding of metal fragments following 30 passes through human integument. Preliminary investigations utilizing mass spectrometry and X-ray analysis unveiled a higher concentration of nickel and chromium residues in fresh human tissue when using cutting needles compared to tapered ones. Moreover, this disparity appeared to be exacerbated when the triangular cutting needles were bent, a common occurrence during routine suturing. These early findings suggest that the use of triangular (cutting) needles may result in the deposition of potent allergens during surgical procedures, potentially leading to severe inflammation. Additionally postoperative wound infection risk would be elevated as there are foreign bodies left behind in the tissue. Tapered needles could prove safer to use in patients in general and especially those who suffer from nickel and or chromium allergies.

Comprise, include, and/or plural forms of each are open ended and include the listed parts and can include additional parts that are not listed. And/or is open ended and includes one or more of the listed parts and combinations of the listed parts.

One skilled in the art will realize the subject matter can be embodied in other specific forms without departing from the spirit or essential characteristics thereof. The foregoing embodiments and the following Examples are therefore to be considered in all respects illustrative rather than limiting of the subject matter described herein.

### EXAMPLES

### Example 1: Tapered Needles Cause Less Tissue Damage than Reverse Cutting Needles: an Experimental Inquiry

Abstract: In this example, a series of biomechanical studies were conducted examining differences between tapered and reverse cutting surgical needles. An in vitro cardiovascular model utilizing porcine aortas demonstrated that reverse cutting needles causes more fluid leakage than tapered needles when comparing needles with maximum diameters of 0.41 vs 0.38 mm (reverse cutting vs tapered), 0.64 mm, and 0.93 mm (p<0.001, p<0.001, p<0.001 respectively). Additionally, reverse cutting needles involved greater amounts of tissue than taper cutting needles of equal size when examined microscopically. The study showed that taper needles may cause less damage to tissue than reverse cutting needles.

Introduction: Every suture needle shares three anatomical features: the eye, the body, and the point. Each of these subunits has several different options regarding size and shape, resulting in many distinct needle varieties. The eye may be looped or swaged to attach the suture thread, the body can be curved to various degrees and vary in size and thickness, and the point can be one of several specifically engineered shapes. The exact configuration of suture needle subunits depends on the qualities of the tissue to be sutured, so the surgeon must be cognizant of their needle selection.

Many needle characteristics and their effect on tissue are already well-documented. The needle-suture junction is well-studied, and it is agreed that swaged needles, which crimp the end of the needle around the suture material, are superior to eyed needles due to their convenience, reliability, and reduced trauma to tissue. Furthermore, efforts are being made to bring the needle-to-suture width ratio as close to 1 as possible, to maximize the degree to which the suture material will plug the hole left by the needle, reducing bleeding. Similarly, the theoretical basis of needle body curvature and needle size selection is well-understood, largely dependent on the thickness of the necessary suture material, maneuverability required for proper suturing technique, as well as the surgeon's preference.

Needle point geometry can mainly be divided into either a cutting-edge or a taper point. Curved cutting-edge needles have at least two opposing cutting edges aligned parallel to the needle's axis of rotation and can be further subdivided into two main categories: conventional and reverse cutting. The former has a third cutting edge along the interior arc of the needle, producing a triangular cross-section at the tip. However because of this design, conventional cutting needles pose the risk of tissue tear out as the inside edge can easily cut up and toward the edges of the wound as the needle is rotated. Reverse cutting needles instead place the third cutting edge along the outside arc of the needle, keeping the triangular cross section but theoretically reducing the risk of tissue tear out. Due to this, reverse-cutting needles are generally favored to conventional cutting needles. The tips of both cutting-edge varieties extends to the maximum diameter of the needle, at which point the body becomes round. On the contrary, taper point needles are round-bodied along their entirety, displaying a circular cross-section at the tip. Various sources state that tapered needles are appropriate to use in cases where tissue is delicate and friable, because they pierce and spread tissue apart, rather than slicing like cutting-edge needles. However, there is a paucity of empirical evidence to support this claim. Therefore, the purpose of this study was to quantitatively compare taper point and reverse cutting needles and examine the effect of these two needle point geometries on tissue.

### Methods:

### Ex Vivo Cardiovascular Model to Simulate Bleeding

The leakage rate caused by reverse cutting and taper-point needles of various diameters was compared (Table 1). A pulsatile ex vivo model of a cardiovascular system was constructed to model tissue damage to blood vessels caused by needle passage. The model used a programmable motor and a series of reservoirs and tubing to pulse fluid at a rate approximating standard pulse rates and at a pressure of 120/80 mmHg to mimic clinical conditions. Water was used to model the fluid dynamics of blood. The model contained a segment of tubing that was removable and interchangeable with porcine aortas (AORTA SUPPLIER). Reverse cutting needles had diameters of 0.41 millimeters (mm), 0.64 mm, and 0.93 mm. Taper point needles had diameters of 0.38 mm, 0.64 mm, and 0.93 mm. Reverse cutting and taper point needles were paired into groups of small (0.41 mm and 0.38 mm), medium (0.64 mm) and large (0.93 mm).

TABLE 1
Descriptive variables of different types of surgical needles used.

| | | | Diam- | | |
|---|---|---|---|---|---|
| | Needle | Point | eter | | |
| Maker | Type | Geometry | (mm) | Curve | Grouping |
| Ethicon * | RB-1 | Taper | 0.38 | ½ c | Small |
| Ethicon * | P-3 | Reverse Cutting | 0.41 | ⅜ c | Small |
| Ethicon *† | SH | Taper | 0.64 | ½ c | Medium |
| Sharpoint *† | FS | Reverse Cutting | 0.64 | ⅜ c | Medium |
| Ethicon * | CT-2 | Taper | 0.93 | ½ c | Large |
| CP Medical | NCP-1 | Reverse Cutting | 0.93 | ½ c | Large |

Porcine aortas were attached to the tubing of the fluid circuit and were observed for any leakage at the attachment points. Each aorta was pierced by two needles of similar diameters (one reverse cutting and one taper pointed) with suture thread removed at the base. Aortas were punctured by passing the needle into the aortic cavity and back out again, resulting in one entry and one exit perforation. Curved needles were uniformly rotated about the center of the needle's curvature. Fluid leakage rate was measured by collecting and weighing all fluid that dripped from the puncture sites over the course of one minute. The weight of the water was converted from grams to milliliters at a (1 milliliter/1 gram) conversion rate. Data collection was repeated 5 times for every puncture event, and the average of the five trials was used for statistical analysis. System fluid pressure was monitored and held at a constant 120/80 mmHg across all five trials.

Aortas were first punctured by reverse cutting needles and measurements were taken as described. The puncture sites were then clamped with hemostats and the aorta was carefully monitored for two minutes to ensure that no fluid was leaking at any point along the aorta. The system was re-checked to ensure that the fluid pressure remained at 120/80 mmHg. The aorta was then pierced by a taper point needle of equivalent diameter and fluid leakage was monitored. Aortas were discarded after being pierced by a single pair of reverse cutting and tapered needles, and fresh needles were used for every piercing. Trials were repeated with ten aortas for each needle diameter 0.38 mm, 0.64 mm, and 0.93 mm.

### Histological Analysis

The effect of taper point versus reverse cutting needle point geometry on skin tissue was observed on a histological level. Donor human skin tissue was harvested following an abdominoplasty. Tissue was defatted to create uniform samples of dermis and overlying epidermis measuring 1.0 centimeter (cm)×1.0 cm. Samples were placed on an elevated surface with a small gap in the center and were oriented so that the center of the sample was directly overlying the center of the gap. The edges of the sample were held in order to secure it.

One reverse cutting needle and one taper point needle of equivalent needle diameter (0.64 mm) were selected (Table 1). The reverse cutting needle was inked by fully submerging the needle in green tissue marking dye (Mark-It Tissue Marking Dyes, ThermoFisher Scientific, Waltham, MA) and the taper pointed needle was inked with red tissue marking dye. Excess ink was allowed to drip off. Inked needles were then passed through the center of the elevated tissue sample through the underlying gap and excess ink was patted dry with gauze. Each sample was pierced by both needles. The piercing order was randomly assigned so that half of the samples were first pierced by the reverse cutting needle and half by the taper pointed needle. A total of 10 tissue samples were used for experimentation.

Samples were then processed in frozen sections. Samples were embedded face down in a tissue freezing medium (Cryochrome Embedding Resin, Epredia, Kalamazoo, MI) and cut to a thickness of 7 microns with a cryostat (Leica CM1850 Cryostat, Leica Biosystems, Deer Park, IL). Great care was taken to orient the samples so that sections would be taken in parallel to the tissue face. The first section was taken after cutting 70 microns deep into the plane of the tissue. Four sections were taken per tissue sample, each 70 microns apart in depth. Sections were stained with a standard hematoxylin and Eosin-Y (H&E) stain and viewed at −40× magnification. Once staining was complete, sections were mounted with a xylene-based mounting medium (Mounting Medium, Richard-Allan Scientific, Kalamazoo, MI) and were scanned for red and green ink. Tissue sections were verified to have only a single locus each of red and green ink.

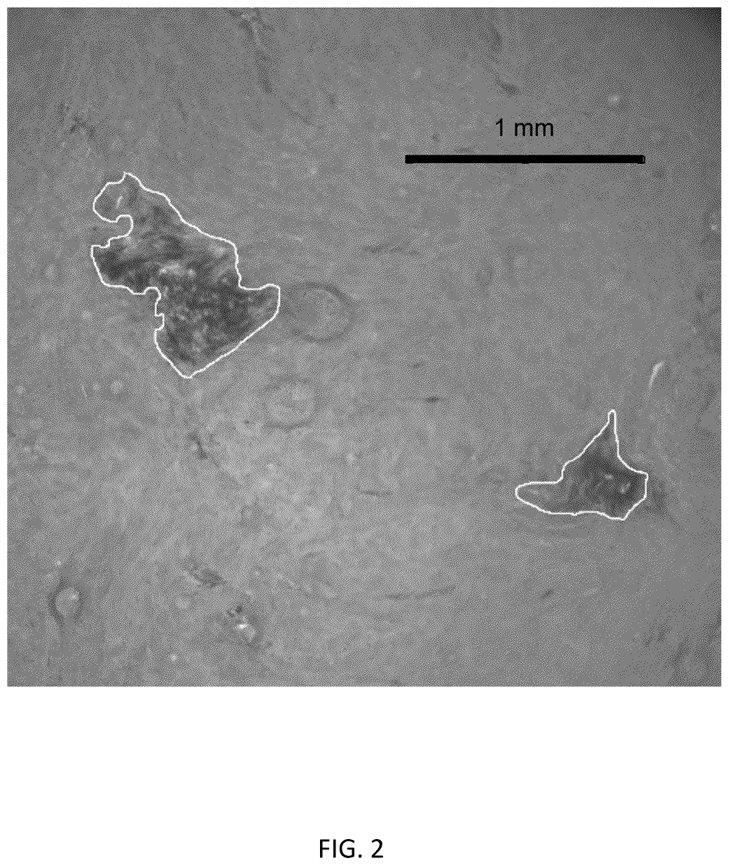

Photographs were taken of each section and uploaded to ImageJ for analysis. Tissue containing green and red ink were digitally outlined and the contained area was recorded for both colors (FIG. 2). Forty total sections were processed and analyzed. Measurements was discarded from two sections because tissue was torn during frozen section processing, rendering digital analysis impossible. In total, 38 measurements were analyzed for both red and green dye. For both experiements, statistical differences among and between groups were assed using a one-way ANOVA and t-tests, with statistical significance determined by p<0.05. All data was analyzed in R.

### Results:

Taper Point Needles Cause Less Porcine Aortic Leaking than Reverse Cutting Needles.

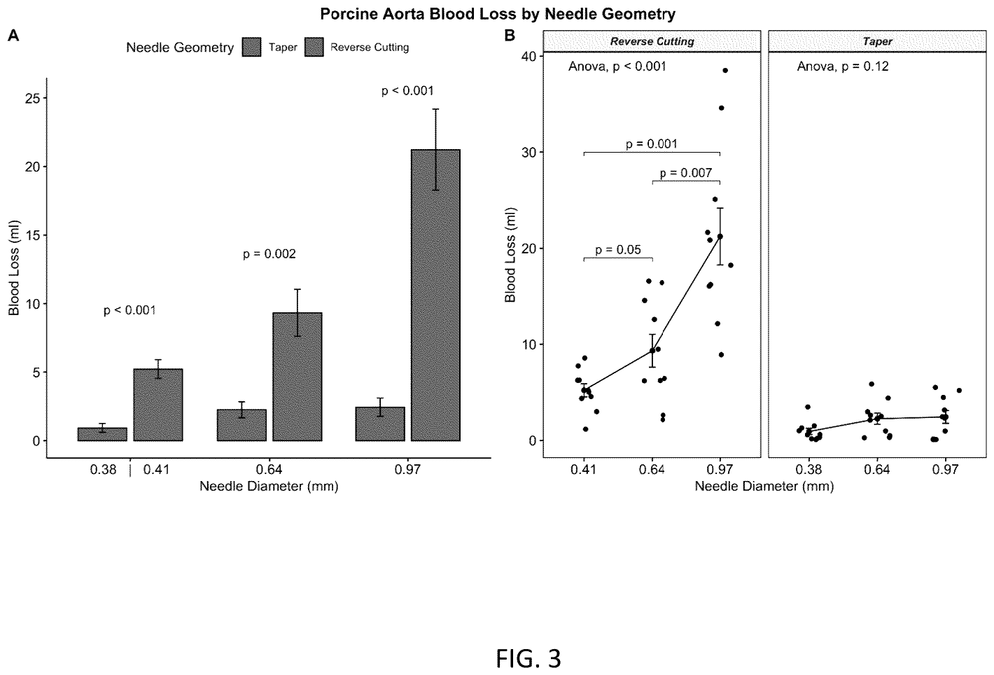

For small, medium and large sizes, tapered needles caused the aorta to leak at a rate of 0.93 mL/min, 2.3 mL/min, and 2.0 mL/min, while reverse cutting needles caused the aorta to leak at 5.22 mL/min, 9.3 mL/min, and 21 mL/min, respectively (FIG. 3). Statistically significant differences were detected between reverse cutting and taper needles at all sizes. Additionally, Reverse cutting needles demonstrated more leaking as needle size increased (Anova, p<0.001), but taper needles did not (Anova, p=0.12).

Taper Point Needles Involve Less Tissue than Reverse Cutting Needles.

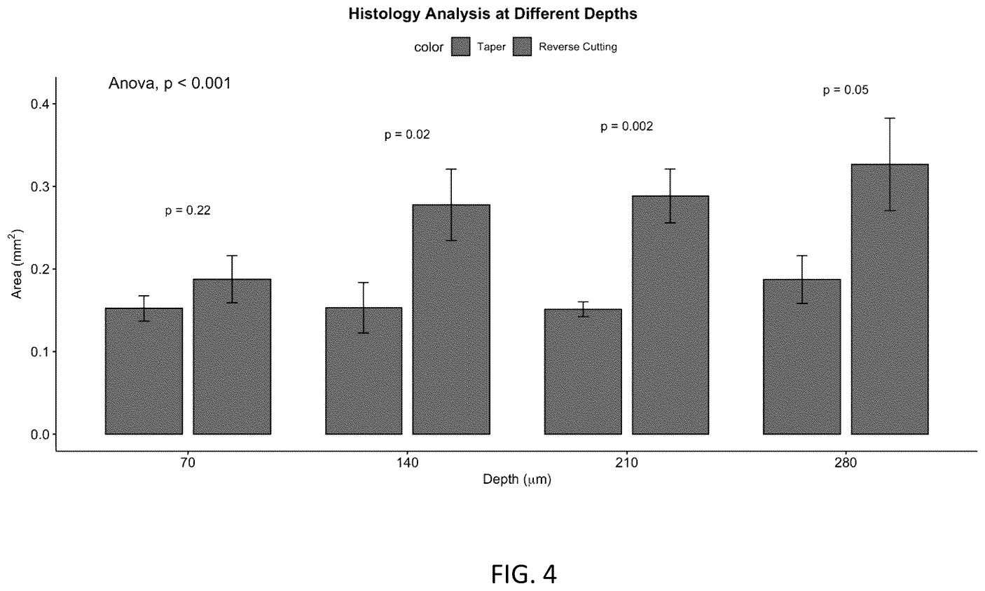

Taper pointed needles resulted in more dye involvement of tissue than reverse cutting needles did (FIG. 4). At 70 microns below the surface of the epidermis, the reverse cutting needle involved 1.24 times more area than the tapered needle, although a statistically significant difference was not achieved. At 140, 210, and 280 microns deep, the reverse cutting needle respectively involved 1.82, 1.91, and 1.81 times more area than the tapered needle; a statistically significant difference was detected at 140 and 210 micron depths (FIG. 4). Pairwise t-tests between depths for both reverse cutting and taper needles revealed no significant differences.

Discussion: The ideal surgical needle varies based on many factors, but at minimum will have the appropriate specifications to induce minimal tissue trauma while also allowing efficient suturing by the surgeon. However, the base of evidence upon which needles are selected remains unclear, particularly regarding needle point geometry. A 2003 study by Lam et al. proposed that choice of needle has no effect on porcine tendons despite the fact that traditionally, round-bodied needles are used in tendon repair surgery. However, a more recent 2021 study by Zolotov et al. indicates that reverse cutting needles offer 40% greater tensile strength in tendon repairs than conventional cutting counterparts, suggesting that needle point geometry selection is highly consequential. This implication is reflected in modem justification of using taper point needles, which are generally thought to minimize tissue damage. This study aims to compare the effect of taper and reverse cutting needles to quantify the different effects of the two needle point geometries on tissue.

The cardiovascular model was designed to demonstrate differences in tissue damage with the assumption that greater damage to the walls of the aorta will result in greater water leakage rates. Measurements for tapered needles were conducted after testing of reverse cutting counterparts so that if puncture marks left by reverse cutting needles could not be stopped from leaking, leakage recorded for taper needles would be overestimated. However, the authors noted no difficulty closing the puncture sites from reverse cutting needles. Furthermore, despite this adjustment, tapered needles outperformed reverse cutting needles at all tested sizes, and this difference was exaggerated as needle diameters increased. Interestingly, reverse cutting needles showed higher leakage rates as needle diameters increased, but tapered needles did not. This observation may support the generally accepted hypothesis that while cutting-edge needles slice tissue, tapered needles separate and spread tissue apart. Under this explanation, as cutting-edge needles get larger, they would transect greater amounts of the fibers composing the aorta wall, greatly compromising the vessel's waterproof nature. However, taper point needles would minimize transection of these fibers and instead push them apart during needle passage. Once the tapered needle has fully passed through the aorta, the fibers may realign to their original configuration, whereas the cutting-edge needle would render the fibers damaged, compromising local structure.

Microscopic imaging of the histological effect of the two needle varieties further indicates that there is an inherent difference in their mechanisms. If the two needles functioned identically, they should theoretically affect equivalent areas of tissue. However, the reverse cutting needle involved more tissue than the tapered needle did. Notably, there was not a statistically significant difference at 70 microns below the surface of the epidermis. Because the thickness of the epidermis on the trunk is typically between 60 and 80 microns, results may have been skewed by the epidermal/dermal junction, as the two layers of the skin require different amounts of force to pierce.

It is possible that the different mechanisms of reverse cutting and tapered needles may influence their ease of use. One of the key factors that increases the usability of a surgical needle is its ability to penetrate tissue. Thacker et. al identified the length of the cutting edge as a major contributor to needle sharpness, which influences its penetrative capability. Concordantly, a study by Hoard et al. states that the maximum required penetration force of the reverse cutting needles was significantly lower than tapercutting variants, which possess substantially shorter cutting edges. Despite this, the authors did not notice any increased difficulty when using tapered or reverse cutting needles during testing or during clinical use.

Minimization of tissue damage carries significant implications and can reduce operative bleeding, influence scarring outcome, and improve infection rates. Although a statistically significant difference between the tissue damage caused by reverse cutting and tapered needles was observed, the above experiments are currently unable to comment on the magnitude of said differences in practical clinical settings. However, while the above experimental setups do not sufficiently emulate clinical conditions to draw such conclusions, they highlight that the two needle point geometries exhibit a significant difference. Over the course of an entire operation and dozens of needle punctures, such differences may manifest different surgical outcomes, although additional clinical study is required.

Conclusion: Tapered surgical needles cause the ex vivo cardiovascular model to leak at a slower rate and involves less tissue on a microscopic level than reverse cutting counterparts, indicating that tapered needles cause less tissue damage.

### Example 2: Correlation of Suture Needle Tip Geometry with its Ability to Cut Through an Artificial Skin Model

Introduction: It is generally believed that the design of a suture's needle impacts its ability to tear through the skin. For example, reverse cutting needles are generally preferred over cutting needles, as their design is thought to distribute the tension more evenly along the suture, thereby reducing the likelihood of a concentration of tension at the suture's entry and exit points. This reduced tension can lead to less damage to the skin tissue, and consequently, less chance of the suture material cutting through the skin.

Recently, Xiong and Bennett used a force gauge to investigate the correlation of suture diameter and its ability to cut through an artificial skin model. While performing a suture tear-through test on sheets of silicone, they noted an interesting phenomenon—(1) the suture causes the skin substitute to bunch; (2) a small tear occurs; (3) more bunching occurs; and (4) a complete tear-through occurs. This suggests that any defect or trauma caused to the artificial skin model facilitates further tearing until a complete tear-through occurs. Since different needle types cause different types and levels of trauma when passed through the skin, they should impact suture's ability to tear through the skin. Thus, this study sought to test the relative ability of cutting, reverse cutting and taper needle sutures to cut into and completely tear through an artificial skin model that simulates the human skin.

Experimental Methods: This study investigated 5-0 polypropylene (Prolene; Corza Medical, MA) with 13 mm ⅜ circle conventional cutting, reverse cutting and taper needles. For each suture needle type, the wire diameter was measured by an eyepiece micrometer with the microscope.

Adapting the experiment protocol performed by Xiong and Bennett (2021): Each suture needle was passed through a 1-mm thick silicone sheet skin substitute (WormTattoo Practice Skin; Amazon, Portland, OR) 5 mm from its edge and then knotted with a square knot to form a loop. The other side of the loop was attached to a digital force meter (Nextech DFS50; Nextech Global Co., Bangkok, Thailand) mounted on a horizontal manually cranked test stand (APW Test Stand, Amazon, Portland, OR). The digital force meter recorded on a computer a force-time curve when pulled manually at a constant rate using the test stand. This procedure was repeated 10 times for each suture needle type. The mean force in newtons was calculated by averaging data points across the flat part of the curve (maxima), representing the force at which the suture was cutting into and then tearing the skin substitute.

Results: Using a microscope micrometer, the needle diameter for both reverse cutting and cutting needles measured 0.48 mm, consistent with manufacturer specifications. Surprisingly, the only 13 mm taper needle offered for 5-0 polypropylene had a much smaller diameter (0.30 mm).

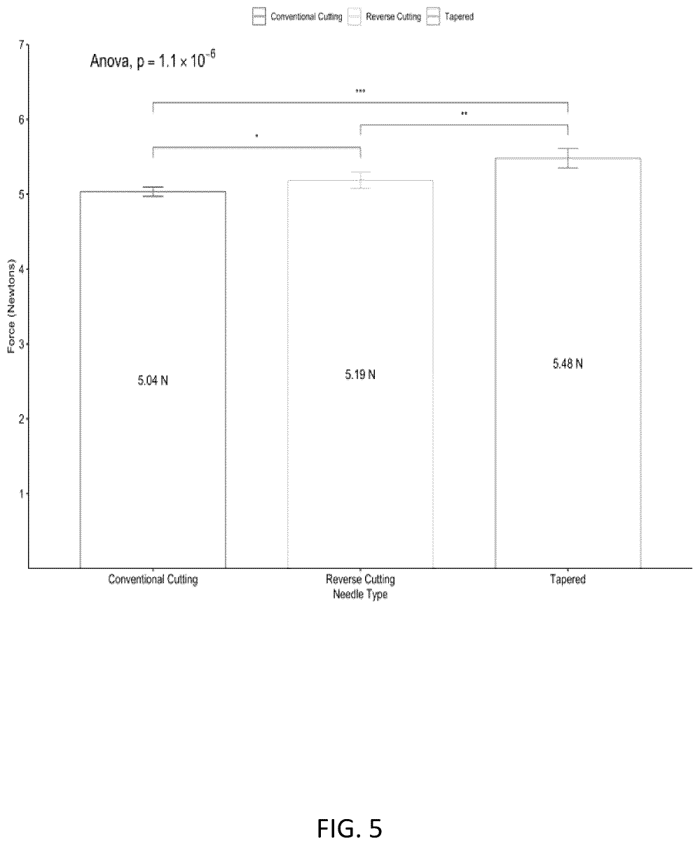

The average force required to tear through the artificial skin model was 5.04, 5.19 and 5.48 Newtons for cutting, reverse-cutting, and taper needles, respectively. A one-way ANOVA revealed a global statistical difference (p<0.001), and pairwise student t-tests showed significant differences between each combination of needle geometry (FIG. 5). Interestingly the difference between cutting and reverse cutting needles (0.15, p<0.05) was smaller than that between reverse cutting and taper needles (0.29, p<0.01).

Discussion: study results show that taper needles allow for a greater force to be applied to the dermis before causing a tear through event, compared to cutting needles. One possible mechanism is that the geometry of the defect in the case of taper needles is circular, and thus when tension is applied it is more evenly distributed compared to its cutting counterparts (FIG. 6). Interestingly, reverse cutting needles showed a similar advantage over cutting needles, albeit much smaller. By observing and comparing the patterns of tearing, the study noticed that reverse cutting needles rarely tore the material in a straight line toward the skin edge (as conventional cutting needles did). Rather the tears were always off to one side. One possible mechanism for this is that any force applied to the suture, which is not perfectly perpendicular to the skin edge, forces the suture material to one of the corners of the triangular shaped defect. Consequently, providing a weak spot, by which the material can more easily tear through the skin.

Conclusion: the study demonstrated that compared to conventional cutting and reverse cutting needles, tapered needles allow for more force to be applied to an artificial skin matrix before ripping through.

### Example 3: Translational Cartilage Suture Tear Through Model Comparing Reverse Cutting and Tapered Point Needles

Introduction: Non-melanoma skin cancers frequently present on the auricle and the nose, occurring in approximately 62.8% of cases on the head and neck area. The resulting defect following excision may heal by secondary intention or may necessitate flaps, grafts, and or reconstruction involving cartilage. The intricacies of ear reconstruction, with its three-dimensional nature, pose a formidable challenge for cutaneous surgeons. During ear reconstruction, manipulation and suturing of the cartilage may be necessary, and due to the complex anatomical relationships, even minor defects like cupping or notching may lead to disfigurement. Likewise, the nose, as a conspicuous facial feature, poses a formidable challenge for reconstruction, particularly when cartilage is involved. As a result, achieving the ideal aesthetic outcome may necessitate multiple micro-adjustments during surgery. Yet, the iterative nature of needle punctures or the passage of sutures through the cartilage may lead to its fragmentation, adding undesirable complexity to the reconstruction process. Hence, the meticulous choice of sutures, especially needles, is a matter of paramount importance. When considering needle selection, needle point geometry emerges as a pivotal factor, categorized into cutting and tapered needles. Cutting needles feature three cutting edges that triangulate, forming a triangular cross-section, while tapered needles gradually narrow into a single point, resulting in a circular cross-section. In the context of cutting needles, the third cutting edge is located on the interior arc facing the wound, whereas the reverse cutting needle positions its third cutting edge on the exterior arc, away from the wound.8 Cutting needles, with their sharp edges, slice through tissues, whereas tapered needles push tissues apart as they traverse through.

Consequently, tapered needles inflict less tissue damage compared to cutting needles. Despite the prevalence of reverse cutting needles in cutaneous surgery, particularly when suturing cartilage, there exists a dearth of research on the mechanics of needle point geometry concerning cartilage. In light of this, the present study aimed to evaluate the force required to tear through a cartilage model using two different needle geometries and three distinct needle diameters. It is hypothesized that reverse cutting needles would require less force to tear through the cartilage compared to taper point needles.

Methods: Denuded rabbit ears were secured on one edge to mimic human ear cartilage attachment. Sutures were passed through the cartilage 3 millimeters (mm) from the edge of the sample and tied to create a 15 mm loop; the loop was then attached to the force gauge (Nextech DFS50; Bangkok, Thailand) using a clamp. All sutures were placed by the same investigator (M.E.M). United States Pharmacopeia (USP) size 2-0, 3-0, and 5-0 polypropylene with a small half circle (SH; Ethicon; Bridgewater Township, NJ), circle taper-2 (CT-2; Ethicon; Bridgewater Township, NJ), precision reverse cutting (PS-3; Sharpoint; Westwood, MA), and taper point baby blue needle (BB; Sharpoint; Westwood, MA), 2-0 Nylon with a for skin needle (FS; Sharpoint; Westwood, MA) and 0-0 polydioxanone monofilament with NCP-1 (Covetrus; Portland, ME) needles were evaluated. Needle body diameters were measured with a digital caliper (Husky; Ontario, CA). Reverse cutting and taper point needles were grouped into small (0.36 mm and 0.45 mm), medium (0.75 mm and 0.60 mm), and large (1.11 mm and 1.06 mm) diameter groups (Table 2).

TABLE 2
Description of sutures utilized in cartilage tear force experiment.

| | Suture | | | USP | Diameter | Curve | |
|---|---|---|---|---|---|---|---|
| Manufacturer | Material | Needle | Point Geometry | Gauge | (mm) | (c) | Group |
| Sharpoint | Prolene | BB | Taper | 5-0 | 0.45 | ⅜ | Small |
| Sharpoint | Prolene | PS-3 | Reverse Cutting | 5-0 | 0.36 | ⅜ | Small |
| Ethicon | Prolene | SH | Taper | 2-0 | 0.60 | ½ | Medium |
| Sharpoint | Nylon | FS | Reverse Cutting | 2-0 | 0.75 | ⅜ | Medium |
| Ethicon | Prolene | CT-2 | Taper | 3-0 | 1.06 | ½ | Large |
| Covetrus | Poly-Dox | NCP-1 | Reverse Cutting | 0-0 | 1.11 | ½ | Large |

Using the force gauge attached to the suture, the investigator pulled the suture at a constant rate until either tear through of the cartilage or suture material failure occurred. The resulting force-displacement graphs were generated, and the maximum force required was recorded. Ten trials of each suture type were conducted. Statistical analyses were performed using the statistical package R (The R Foundation; Vienna, Austria). The collected results were averaged (mean±SD), and the significance level was set to 95% confidence with p≤0.05.

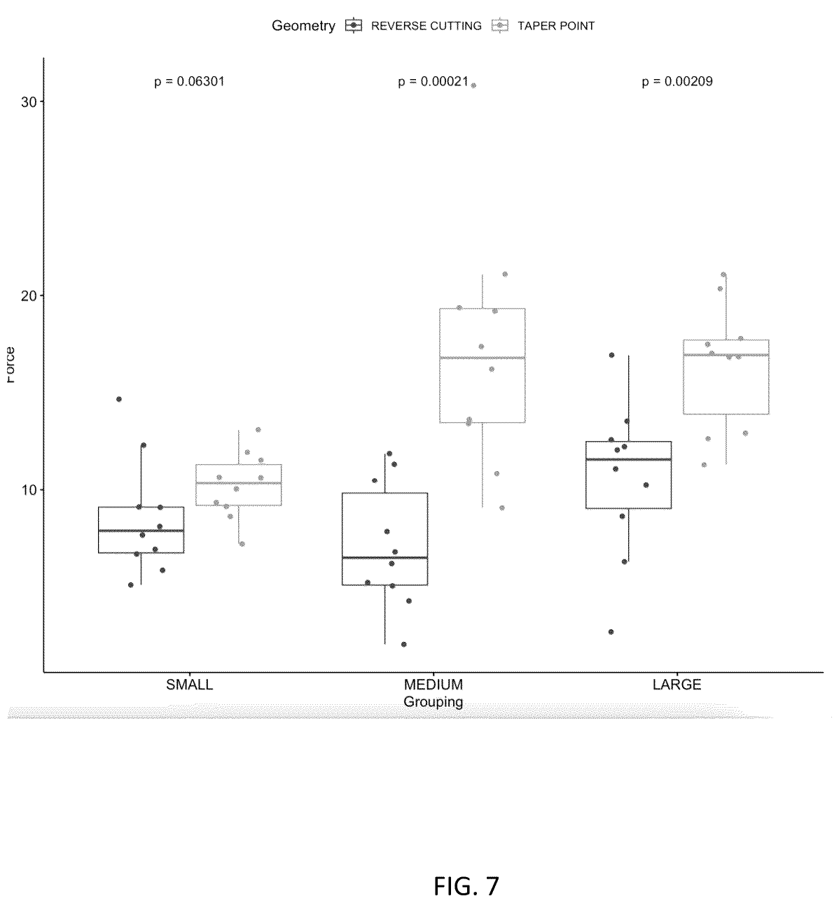

Results: The mean tear through force for PS-3, BB, FS, SH, NCP-1, and CT-2, was 8.57, 10.24, 97 7.13, 17.11, 10.64, and 16.44 newtons (N), respectively (Table 3). For medium and large diameters, the suture completely tore through the cartilage in all trials. At the small diameter, the taper point needle's suture failed before tearing through the cartilage in all but one trial. Conversely, all other trials resulted in tearing the suture through the cartilage. PS-3 sutures required less force to tear through cartilage than BB sutures (p=0.063), FIG. 7. FS required significantly less force to tear through cartilage compared to SH (p<0.001), FIG. 7. NCP-1 required significantly less force to tear through cartilage compared to CT-2 (p<0.001), FIG. 7.

TABLE 3
Results for each needle diameter group for both reverse cutting and
taper point and the mean force required to tear through cartilage.

| | | Mean | | Percent |
|---|---|---|---|---|
| Geometry | Grouping | (N = 10) | SD | Difference |
| Reverse Cutting | Large | 10.64 | 3.98 | |
| Taper Point | | 16.44 | 3.22 | +54.44% |
| Reverse Cutting | Medium | 7.13 | 3.24 | |
| Taper Point | | 17.11 | 6.18 | +139.83% |
| Reverse Cutting | Small | 8.57 | 2.95 | |
| Taper Point | | 10.24 | 1.73 | +19.45% |

Discussion: Reconstructing cartilaginous structures, such as the ear and nose, poses a considerable challenge for cutaneous surgeons, particularly when cartilage shredding occurs as the result of suturing. The pivotal aspect of successfully navigating such surgical maneuvers lies in the meticulous selection of sutures, with a pronounced focus on the geometry of the suture needle point. Although the reverse cutting needle is the prevailing choice in dermatologic and plastic surgery, its application in cartilage suturing warrants further investigation.

In the present study, it was found that taper point needles demanded more force than reverse cutting needles to tear through cartilage at all three diameters, with a trend toward more force to tear through the cartilage with increasing needle diameter size. Because taper point needles theoretically push tissues apart instead of lysing tissues as with cutting needles, the structural integrity of the cartilage is maintained and necessitates more force to pull the suture through the tissue. Conversely, the triangular wound made from the reverse cutting needle facilitates tearing and fracturing from one of two of the triangular apices facing the wound resulting in less force required to tear through the cartilage. In the context of cutaneous surgery, where precision is paramount, the study findings challenge the status quo of using reverse cutting needles for suturing cartilage. The results suggest that the adoption of taper point needles for suturing cartilage minimizes tissue damage, thus, maintaining the structural integrity of the cartilage and may enhance the overall success of the surgery.

Furthermore, younger cartilage exhibits greater robustness and resilience than its older counterparts. Aging cartilage, characterized by diminished elastin, cell density, and glycosaminoglycans, tends to be more brittle and non-pliable. Additionally, increased non-enzymatic glycation with age further contributes to increased cartilage stiffness. Consequently, a smaller taper needle, such as a TF, may be more suitable when working on smaller nasal cartilage. Although not all the sutures were made of the same material, Xiong and Bennett found that there was no difference in tear through force when comparing different suture types. This finding enabled this study to isolate and emphasize the specific influence of needle point geometry mechanics on cartilage, independently emphasizing its crucial role in surgical decision-making.

### Example 4: Exploring the Impact of Needle Geometry on Tissue Damage: A Combined Analysis of Human Skin and Porcine Cardiovascular Models

Introduction: The suture needle plays a pivotal role in the suturing process, serving as a key determinant of success by facilitating precise tissue alignment while minimizing trauma to the surrounding tissue. Choosing the optimal surgical needle for a specific procedure hinges on numerous factors, given that the ideal needle's properties vary according to the characteristics of the tissue being sutured. The suture needle is made of three distinct regions: shank, body, and point, each offering a spectrum of designs, shapes, and sizes that result in unique needle subtypes.

Among the needle point geometries are the cutting-edge and the tapered point (TP). The cutting-edge needle features a triangular cross-section at its tip, extending to the maximum needle diameter point where the body becomes round, enabling the needle to effectively slice through tissue. This category further divides into conventional cutting and reverse cutting (RC) needles. RC needles, with two opposing cutting edges aligned parallel to the needle's axis and a third cutting edge along the exterior arc, theoretically reduce the risk of tissue tearout compared to conventional cutting needles. Consequently, RC needles are generally preferred to conventional cutting variants. In contrast, TP needles exhibit a round-bodied structure from base to tip and are proposed for delicate friable tissues. Unlike cutting-edge needles that slice through tissue, TP needles pierce and push tissue apart.

The suturing process inherently disrupts surrounding tissues, causing cumulative damage that can result in permanent structural defects and potential postoperative complications such as bleeding and infection. Various needle characteristics, including size, diameter, length, geometry, suture, and needle-to-suture diameter ratio, influence the extent of iatrogenic tissue trauma. The combination of properties across the needle components has led to a myriad of suturing needles, posing a challenge in determining the optimal choice.

Previous researchers have explored the impact of needle point geometry on iatrogenic tissue damage to help clarify this decision. In one set of experiments using a canine model, simple interrupted sutures were placed to close enterotomy incisions, and methylene blue solution was infused to assess leakage rates correlating with tissue damage. The results showed no significant difference in fluid leakage rates between TP and RC needles. However, the researchers acknowledged a potential confounding factor, the needle-to-suture diameter ratios nearing zero, which was not considered in the model. It is believed that no other studies have explored the effect of needle geometry on iatrogenic tissue damage.

This study seeks to build upon prior research by providing a more nuanced understanding of the secondary effects of using TP and RC needles. The authors developed a pulsatile, ex vivo model utilizing aorta tissue, quantitatively comparing fluid leakage rates when puncturing the aorta with three diameters of TP and RC needles. Differences in tissue damage are explored under the premise that greater damage to the aorta walls leads to increased fluid leakage rates. Additionally, the study examines the histological impact of TP and RC needle point geometries on human skin tissue at four depths. These investigations represent improvements over previous research as they isolate needle geometry and exclude any potential influence from suture material on tissue damage.

### Methods:

### Pulsatile, Ex Vivo, Cardiovascular Model to Simulate Bleeding

A pulsatile, ex vivo, porcine aorta model was constructed to model tissue damage to blood vessels caused by needle passage. The model consisted of a diaphragmatic motor, fluid, reservoirs, and tubing to push fluid at a rate of 70 beats per minute at a pressure of 120/80 mmHg to mimic a standard clinical blood pressure waveform. The motor was controlled by a computer program written in Python (Stichting Mathematisch Centrum, Amsterdam, The Netherlands) that allowed for fine adjustments to motor output and rhythm to maintain consistent waveforms across all experimental trials. Water modeled the fluid dynamics of blood. The model contained a segment of tubing that was removable and interchangeable with porcine aortas (Animal Technologies Inc., Tyler, TX).

Porcine aortas were attached to the tubing of the fluid circuit and observed for leakage at the attachment points. Each aorta was pierced by two needles of similar diameters (one RC and one TP), each with suture thread removed at the base. RC needle diameters were 0.41 millimeters (mm), 0.64 mm, and 0.93 mm, respectively. TP needle diameters were 0.38 mm, 0.64 mm, and 0.93 mm. RC and TP needles were paired into groups of small (0.41 mm and 0.38 mm), medium (0.64 mm), and large (0.93 mm) (Table 1).

Aortas were punctured at orthogonal angles relative to the center of the aorta lumen by passing the needle into the aortic lumen and back out again at the circumferentially opposing side, resulting in one entry and one exit perforation per needle. Needles were uniformly rotated about the center of the needle's curvature. The fluid leakage rate was recorded by measuring the mass of all fluid that dripped from the puncture sites over one minute. The mass was then converted to volume by assuming the density of water as 1 g/mL. Data collection was repeated 5 times for each needle type, and the average of the five trials was used for statistical analysis. System fluid pressure was monitored and held at a constant 120/80 mmHg waveform across all five trials.

The aortas were first punctured by RC needles and measurements were taken as described. The puncture sites were then clamped with hemostats, and the aorta was carefully monitored for two minutes to ensure no fluid leakage. The system was re-checked to ensure the fluid pressure remained at 120/80 mmHg. The aorta was then pierced by a TP needle of equivalent diameter, and fluid leakage was monitored in the described manner. Aortas were discarded after being pierced by a single pair of RC and TP needles; fresh needles were used for every piercing. In total, ten aortas were used for trials of small, medium, and large needle diameters.

### Histological Analysis

The histologic effect of TP and RC needle point geometries on skin tissue was investigated at a microscopic level. Donor human skin tissue was harvested following an abdominoplasty. Tissue was defatted to create uniform samples of dermis and overlying epidermis measuring 1.0 centimeter (cm)×1.0 cm. Samples were placed on an elevated surface with a small gap in the center and oriented so that the center of the sample was directly overlying the center of the gap.

The edges of the sample were secured. One TP needle and one RC needle of equal needle diameter (0.64 mm) were selected (Table 1). The RC needle was inked by fully submerging the needle in green tissue marking dye (Mark-It Tissue Marking Dyes, ThermoFisher Scientific, Waltham, MA), and the TP needle was inked with red tissue marking dye (Mark-It Tissue Marking Dyes, ThermoFisher Scientific, Waltham, MA). Excess ink was allowed to drip off. Inked needles were then passed through the center of the elevated tissue sample through the underlying gap at an orthogonal angle; excess ink on the tissue was patted dry with gauze. Both needles pierced each sample. The piercing order was randomly assigned so that half of the samples were first pierced by the RC needle and half by the TP needle. A total of 10 tissue samples were used for experimentation.

Samples were processed in frozen sections by embedding the sample face down in a tissue freezing medium (Cryochrome Embedding Resin, Epredia, Kalamazoo, MI) and cut to a thickness of 7 microns with a cryostat (Leica CM1850 Cryostat. Leica Biosystems, Deer Park, IL). Great care was taken to orient the samples so that sections would be taken in parallel to the tissue face.

The first section was taken after cutting 70 microns deep into the plane of the tissue. Four sections were taken per tissue sample, all 70 microns apart in depth. Sections were stained with a standard hematoxylin and Eosin-Y (H&E) stain and viewed at 40× magnification. Once staining was complete, sections were mounted with a xylene-based mounting medium and scanned for red and green ink. Tissue sections were verified to have only a single locus each of red and green ink.

Photographs were taken of each section and uploaded to ImageJ (National Institutes of Health, Maryland, USA), an open-source software that measures the pixel intensity of an image, for analysis. Tissue samples containing green and red ink were digitally outlined, and the contained area was recorded for both colors (FIG. 2). Forty total sections were processed and analyzed. Measurements were discarded from two sections because the samples were torn during frozen section processing, rendering digital analysis impossible. In total, 38 measurements were analyzed for statistical data for both red and green dye.

### Statistical Analysis

For all experiments, statistical differences were assessed with ANOVA and t-tests. Statistical significance was designated at p≤0.05.

Results: At small, medium, and large diameters, TP needles caused the aorta to leak at a rate of 0.93 mL/min, 2.3 mL/min, and 2.0 mL/min, while RC needles caused the aorta to leak at 5.22 mL/min, 9.3 mL/min, and 21 mL/min (FIG. 3). Statistically significant differences were detected between TP and RC needles at all diameters. Additionally. RC needles demonstrated more leaking as needle size increased (ANOVA, p<0.001), but TP needles did not (ANOVA, p=0.12) (FIG. 3).

When inked and passed through tissue. TP needles impacted less area than RC needles (FIG. 4), which suggests that TP needles cause less tissue damage. At 70 microns below the surface of the epidermis, the RC needle involved 1.24 times more area than the TP needle, although a statistically significant difference was not achieved (p=0.27). At 140, 210, and 280 microns deep to the epidermal surface, the RC needle involved 1.82, 1.91, and 1.81 times more area than the TP needle, respectively (p=0.01, p=0.002, and p=0.05, respectively) (FIG. 4). When controlled for depth, the TP needle involved less tissue than RC equivalents (ANOVA, p<0.001).

Within the RC needle group, there was no overall statistical difference between the tested depths (p=0.14). There was no overall statistical difference between the tested depths for TP needles (p=0.83).

Discussion: The selection of the optimal surgical needle for a specific procedure is contingent upon various factors, with the primary goal of minimizing tissue trauma while facilitating efficient suturing by the surgeon. Despite the considerable clinical impact of needle choice, the evidence upon which needles are selected in practice remains ambiguous, particularly regarding needle point geometry. For instance, TP needles are commonly used to minimize damage to lumen walls and prevent leakage in anastomotic procedures of vessels and intestines. In contrast, plastic and dermatologic surgery, where minimizing damage to delicate tissue is crucial for aesthetic outcomes, predominantly employ RC needles. This study aims to address the uncertainty surrounding needle selection by comparing the impact of needle point geometry on iatrogenic tissue damage. The assessment includes analyzing the rate of needle hole fluid leak and the total microscopic tissue area affected by TP and RC needles.

The current understanding suggests that cutting edge needles slice through tissue, whereas tapered needles spread tissue apart.6 According to this theory, cutting-edge needles sever increasing amounts of tissue fiber as their diameters increase. Conversely, it is thought that TP needles avoid the transection of tissue fibers, instead pushing them apart during needle passage with minimal structural damage, regardless of needle diameter. Once the TP needle fully passes through tissue, the uncut fibers theoretically realign into their original configurations. The pulsatile, ex vivo, cardiovascular model was designed to highlight the differences in suture needle-derived tissue damage under the premise that greater damage to the walls of the aorta would result in increased fluid leakage rates. The model demonstrated that TP needles caused less fluid leakage than RC needles at all tested sizes and the difference was exaggerated as needle diameters increased, pointing to the underlying mechanistic differences in the two needles.

This inherent distinction was further underscored by the abdominal skin model. TP needles exhibited a propensity for causing minimal tissue damage, affecting significantly less tissue area compared to RC needles at depths of 140, 210, and 280 microns below the epidermis. If the two needles functioned identically, they should affect equivalent cross-sectional areas of tissue. However, RC needles involved as much as 1.9 times more tissue than TP needles, again supporting the notion that RC needles damage local tissue structure whereas TP needles preserve it. In aesthetic and reconstructive surgery, the impact of needle geometries on cumulative damage becomes paramount. Iatrogenic damage not only influences operative bleeding but also exerts a direct influence on scarring outcomes and infection rates. While the experiments mentioned above may not explicitly delve into the precise clinical implications of choosing TP versus RC needles, they do imply inherent differences in functional mechanisms and histological impact. It is crucial to note that throughout the entirety of a surgical procedure, numerous needle punctures occur, and the study models indicate that cumulative tissue damage is more likely when employing RC needles. This underscores the critical importance of thoughtfully selecting needle geometries to mitigate adverse effects and optimize the overall success of the surgical intervention.

It is worth noting that the different functional mechanisms between TP and RC needles may influence their ease of use. A critical factor affecting the usability of surgical needles is their capacity to penetrate tissue. The length of the cutting edge is a major contributing factor to needle sharpness, suggesting that cutting needles have a greater penetrative capacity. Concordantly, the maximum required penetration force of the RC needles is lower than taper cutting variants, which possess shorter cutting edges. Despite this, the authors did not perceive any increased difficulty when using TP instead of RC needles during testing or clinical use.

### Example 5: Illuminating the Mechanics of Surgical Needle Point Geometry

When a suture needle is passed through the skin, the resulting defect is primarily dependent on the geometry of the needle. The prevailing theory in needle point literature suggests that tapered needles separate tissues, while cutting needles cut through tissues as they pass. However, there is no provided reference for this assertion. The assumed distinction in mechanics leads to the hypothesis that tapered needles cause less tissue damage compared to cutting needles. Existing literature supporting this theory relies on the latter part of the “If this, then that” theory of needle point mechanics. Definitive determination of needle point geometry mechanics and confirmation that tapered needles merely push tissues apart remains elusive.

Methods: To explore the needle point mechanics of cutting and tapered needles, this study visualized the damage created by these needle point geometries by puncturing the needle point into a tissue model. The dermis, which is a fibrous structure composed largely of overlapping collagen and elastin fibers to form a matrix, was modeled by a 5-micron synthetic woven matrix (Nugsmasher, Lake Havasu City, AZ) that was secured on all sides to provide tension. Given the size of the woven material, large needles were utilized to clearly visualize their effect. A circle taper-2 (CT-2; Ethicon, Bridgewater Township. NJ) was chosen for the taper point needle, and a reverse cutting needle of similar diameter. NCP-1 (Covetrus, Portland. ME), was utilized to represent cutting needles because reverse cutting needles are most commonly utilized in cutaneous surgery. The taper point needle was then dipped in blue tissue marking dye (Mark-It Tissue Marking Dyes, ThermoFisher Scientific, Waltham, MA) and the reverse cutting needle in yellow dye to determine where the puncture holes were made. Solely the needle points, not the needle bodies, were passed through the model. Then, the defects were inspected using a Leica S6E Stereo Microscope (Leica Microsystems, Deerfield, IL) at 40× magnification.

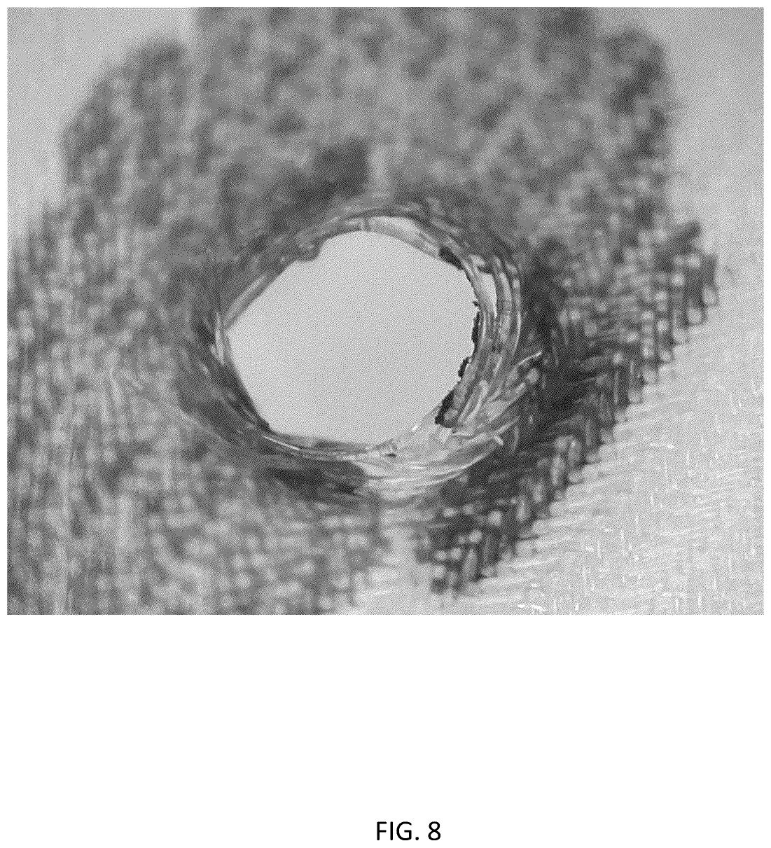

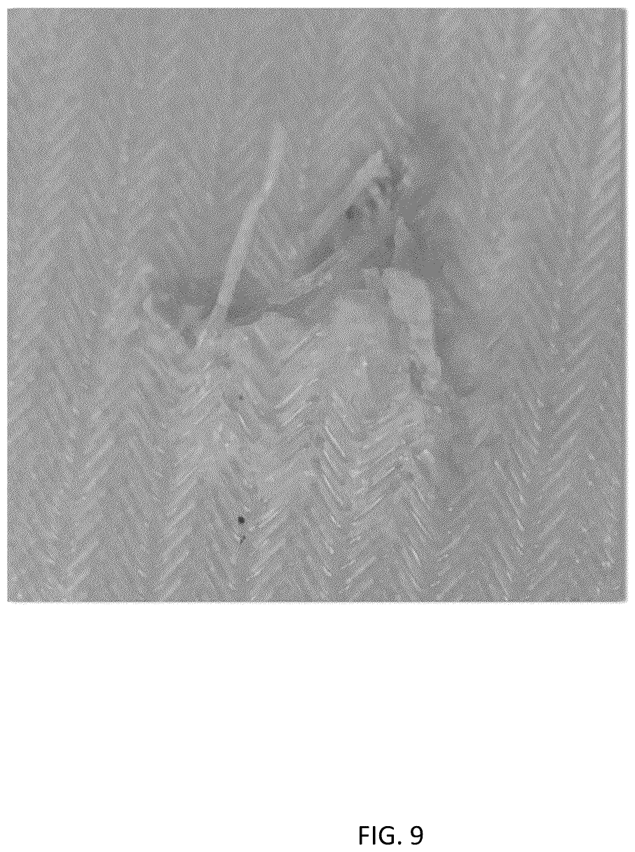

Results: The rounded taper point needle penetration leaves a cylindrical shape in which no mesh fibers have been cut. Instead, the fabric has been pushed in the directions of the force applied (FIG. 8). The reverse cutting needle, with multiple beveled edges, creates a triangular defect, and several transected fibers are visualized at the apex of the needle hole defect (FIG. 9).

Discussion: The results illustrate that taper point needles indeed push tissues apart to create their needle hole defect. The pushing of these fibers created a cylindrical defect, formed by the intact mesh fibers. Translating these results in vivo, tissues are displaced while retaining their structural integrity. Regarding the reverse cutting needle defect, centrally, the mesh fibers are clearly transected. In vivo, this results in tissue lysis and decreased structural integrity. The reverse cutting needle possess three cutting edges, one of which is on the exterior arc of the needle facing away from the wound and is the most commonly utilized needle in cutaneous surgery. Although the cutting needle used was a reverse cutting needle, a conventional cutting needle will produce the same defect with a slightly different orientation with the third cutting are on the interior of the needle.

### Example 6: Impact of Needle Design and Suture Gauge on Tissue Tearing During Skin Suturing: A Comparative Analysis

Introduction: Appropriate needle and suture selection are integral for optimizing wound closure. An ideal suture provides an adequate tensile strength, will tolerate postoperative wound swelling, causes little tissue inflammation, is affordable, and does not disturb surrounding tissue. Needles differ by size, design (cutting or taper), and curvature. Cutting needles have a triangular configuration with three sharp surfaces and are subdivided into conventional cutting needles and reverse cutting needles. In the conventional cutting needle, the sharp surface of the triangle is located on the inside of the needle. The reverse cutting needle is used more often in cutaneous surgery because the cutting edge is located on the exterior arc away from the wound surface. Taper (non-cutting) needles, in contrast, penetrate tissue resulting in tissue spreading, rather than cutting. The tip can be sharp or blunt and the entire body is round. These needle designs are depicted in FIG. 6.

Suture thread materials also vary and factors to consider include the configuration, coating, capillarity, tensile strength, thickness, memory, elasticity, plasticity, and flexibility. The United States Pharmacopeia (USP) designates a number which denotes the tensile or breaking strength of the material. Within a given suture material class, a smaller number denotes a larger caliber, or thicker, suture. The strategic use of finer-caliber sutures on facial regions and thicker counterparts on extremities and the scalp is conventional, with the overarching goal of employing the thinnest possible suture for optimal wound approximation2. However, emerging evidence from select studies, such as that conducted by Xiong and Bennet, challenges this paradigm, revealing that suture thickness correlates with tissue tearing. Their suture retention test on an artificial skin model demonstrated that thinner sutures (5-0) exerted less force in tearing the skin model compared to thicker counterparts (3-0).

Tissue tearing is detrimental because it may damage local structures and increase the risk of wound dehiscence when postoperative swelling occurs. The overall likelihood of tissue tearing while suturing is related to the defect created by the needle, suture thickness, and the load applied to the suture thread. This study aims to scrutinize the impact of needle design (cutting versus taper) and suture thickness on tissue tearing using an artificial skin model.

Materials and Methods: Three needle designs and three suture gauges were evaluated. The precision conventional cutting needle (PC-3; Sharpoint, MA), precision reverse cutting needle (PS-3; Sharpoint, MA) and taper needle with a sharp point (BB; Sharpoint, MA) were compared, all with 5-0 polypropylene suture material. Next, 3-0, 4-0, and 5-0 nylon sutures with precision reverse cutting needles (PS-2; Sharpoint. MA) were tested to compare the effect of suture gauge on tissue tearing.

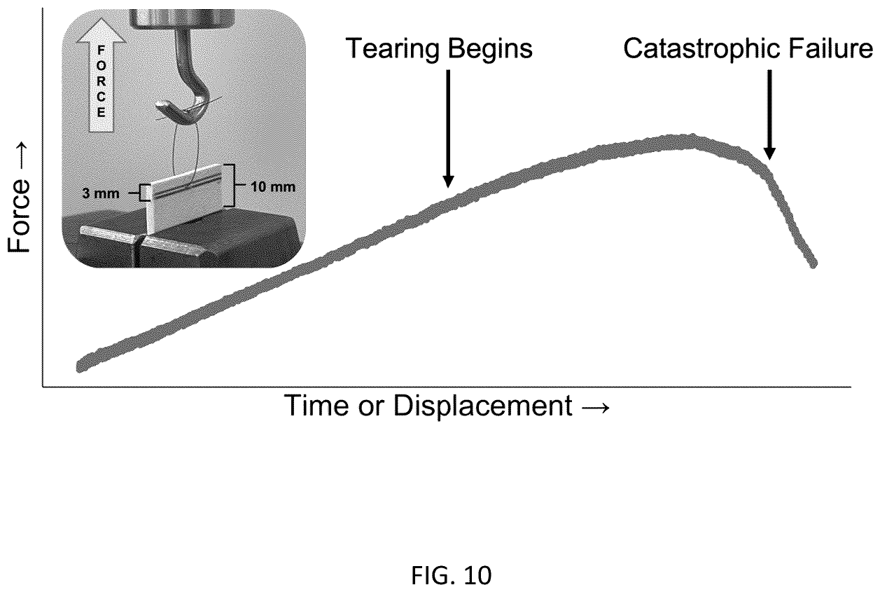

For each needle type and suture gauge, the mid needle diameter was measured with a digital precision caliper micrometer (Mitutoyo; Kanagawa. Japan). A 1.2 mm-thick silicone-based artificial skin sample was secured to a table (Up Tat Supply, Blank Tattoo Practice Skin; Amazon, Portland, OR; FIG. 10). Sutures were passed through the sample 3 mm deep from the artificial wound surface and secured using a sequence of knots to form a 10 mm loop. All sutures were placed by the same investigator. Subsequently, a suture retention test was executed utilizing a professional-grade tensiometer (Instron 6800 Series; Instron, Norwood, MA), applying a force on the suture material at a constant rate (0.1 mm/s) to induce tissue tearing. Force-displacement and force-time curves were generated for each trial (FIG. 10), and the catastrophic failure force, representing the force in Newtons at which the suture completely tore through the skin substitute, was calculated by averaging data points across the curve plateau. This process was iterated ten times for each suture.

Statistical analyses were conducted using the statistical package R (The R Foundation; Vienna, Austria). A one-way analysis of variance analyses (ANOVA) was performed to determine the effect of suture thickness and needle geometry on the mean tear through force. Post-hoc analysis using Tukey's method compared the mean tear through force from suture thickness and suture needle geometry retention testing. Significance was set to a p≤0.05.

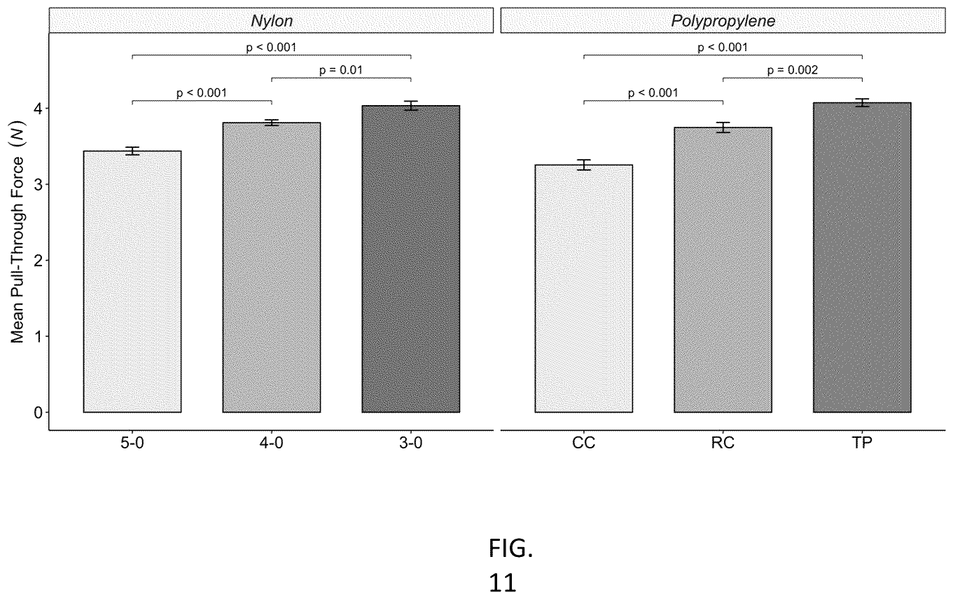

Results: For all PS-2 nylon sutures, the needle diameter measured 0.54 mm (±0.02). For polypropylene sutures, PS-3 and PC-3 cutting needles both measured 0.38 mm (±0.02), while the BB taper point needle measured 0.42 mm (±0.02). Within the PS-2 reverse cutting nylon group, the mean tear through forces were 3.44N, 3.81N, and 4.04N for the 5-0, 4-0, and 3-0 suture sizes, respectively. These results indicate that, while controlling for needle design, larger suture material required more force to cut through tissue (p<0.001). Regarding needle geometry, the mean tear through forces for the PC-3, PS-3, and BB needles were as follows: 3.26N, 3.75N, and 4.07N (p<0.001). Accordingly, when controlling for suture size, the taper point needle required the greatest force to cause tissue damage. Post-hoc analysis using Tukey's method detected significant differences between comparative groups (FIG. 11).

Discussion: In this study, it was observed that sutures with a taper needle required more force to tear through an artificial skin model compared to both types of cutting needles, and sutures with conventional cutting needles required the least force to tear through the model. This suggests that the initial defect created by passing the suture needle and material through the dermis can significantly affect its ability to tear and cause tissue cutting. During suturing, different needle-tip geometries create unique defects (FIG. 6). It is hypothesized that when force is applied to suture occupying different shaped holes (triangular versus circular), the force is distributed differently. When under tension, the suture in the taper needle hole is distributed evenly across the defect due to the cylindrical nature of the needle defect; therefore, there is no weak point or significant fissuring that allows for tissue tears if swelling were to occur. When the same force is applied to suture material occupying a cutting needle defect, the load is disproportionately applied across the triangle such that there are weak points at the apices of the triangle. As a result, less force is required to tear through skin when cutting needles are used.

Taper needles are recognized for causing less tissue damage in various surgical scenarios and are often considered ideal for delicate tasks such as tendon or fascial repair. For instance, in a femoral artery experiment, cutting edge needles were found to strip off endothelial cells from vessel walls, whereas taper point needles were capable of separating intimal folds of a blood vessel causing less overall destruction. The study findings in a skin tissue model align with these observations, suggesting that taper needles can be employed in cutaneous surgery, particularly on the face. Facial skin, being only 1.51-5.14 mm thick, makes the choice of needle particularly crucial during dermal, muscular, or superficial musculoaponeurotic system suturing. The taper needle may offer increased safety and a lower potential for severing nearby blood vessels or damaging nerves compared to cutting needles.

Experts recommend using the smallest caliber suture material to attain adequate tensile strength. Consistent with previous studies, we found that thinner sutures have a greater propensity to cut through the skin. This becomes particularly important in edematous postoperative wounds where absorbable and non-absorbable sutures may be subject to high tension. As suggested by Xiong and Bennett, in delicate areas such as eyelid skin or in severely photodamaged skin, it may be better to use a slightly thicker suture material such as a 5-0 caliber rather than a 6-0 caliber suture to minimize tissue tearing.

The following references provide relevant background on the understanding of those of ordinary skill in the art along with context for citations in the above examples. The content of each of the following is incorporated herein by reference in its entirety:
Ethicon, Inc. Wound Closure Manual, 2005 p. 45-52.
Sergeant. P., Kocharian. R., Patel, B., Pfefferkorn, M., & Matonick, J. (2016). Needle-to-suture ratio, as well as suture material, impacts needle-hole bleeding in vascular anastomoses. Interactive CardioVascular and Thoracic Surgery, 22(6), 813-816.
Lourie, G., Seaber. A., & Urbaniak. J. (1984). Microanastomotic Response to Needle and Suture Size. Journal of Reconstructive Microsurgery, 1(02), 135-138.
Zolotov, A. S., Isokov, S. Kh., & Isokova, A. Kb. (2021). The Optimal Surgical Needle for Tendon Suture: Cutting Edge or Reverse Cutting Edge? Traumatology and Orthopedics of Russia, 27(2), 75-80.
Koyuncuoglu, C. Z., Yaman, D., Kasnak, G., & Demirel, K. (2019). Preference of Suture Specifications in a Selected Periodontal and Implant Surgeries in Turkey. European Journal of Dentistry. 13(01). 108-113.
Bernstein. G. (1985). Needle Basics. The Journal of Dermatologic Surgery and Oncology, 11(12). 1177-1178.
Moy, R. L., Waldman, B., & Hein, D. W. (1992). A Review of Sutures and Suturing Techniques. The Journal of Dermatologic Surgery and Oncology, 18(9), 785-795.
Von Fraunhofer J A, Chu C C (1997) Surgical needles. In: Chu C C, von Fraunhofer J A. Greisler H P (eds) Wound closure biomaterials and devices. CRC Press. Boca Raton, FL, pp 25-38.
Lam. W. L., Garrido. A., Vandermeulen, J., Fagan, M. J., & Stanley, P. R. W. (2003). Cutting or Round-Bodied Needles for Tendon Repair. Journal of Hand Surgery, 28(5), 475-477.
Wagner, G. N., Cooke, S. J., Brown. R. S., & Deters, K. A. (2011). Surgical implantation techniques for electronic tags in fish. Reviews in Fish Biology and Fisheries, 21(1), 71-81.
Gambichler, T., Matip, R., Moussa, G., Altmeyer, P., & Hoffmann, K. (2006). In vivo data of epidermal thickness evaluated by optical coherence tomography: Effects of age, gender, skin type, and anatomic site. Journal of Dermatological Science, 44(3), 145-152.
Frick, T. B., Marucci, D. D., Cartmill, J. A., Martin, C. J., & Walsh. W. R. (2001). Resistance forces acting on suture needles. Journal of Biomechanics, 34(10), 1335-1340.
Towler, M. A., McGregor, W., Rodeheaver, G. T., Cutler, P. V., Bond, R. F., Phung, D., Morgan, R. G., Thacker, J. G., & Edlich, R. F. (1988). Influence of cutting edge configuration on surgical needle penetration forces. The Journal of Emergency Medicine, 6(6), 475-481.
Thacker, J. G., Rodeheaver, G. T., Towler, M. A., & Edlich, R. F. (1989). Surgical needle sharpness. The American Journal of Surgery. 157(3). 334-339.
Hoard, M. A., Bellian, K. T., Powell, D. M., & Edlich, R. F. (1991). Biomechanical performance of tapercut needles for oral surgery. Journal of Oral and Maxillofacial Surgery. 49(11), 1198-1203.
Gantwerker, E., & Hon, D. (2012). Principles to Minimize Scars. Facial Plastic Surgery, 28(05). 473-486.
Bennett R G. Fundamentals of Cutaneous Surgery. St. Louis, MI: Mosby; 1988.
Bennett. Richard G. “Selection of wound closure materials.” Journal of the American Academy of Dermatology 18.4 (1988): 619-637.
Miriam Byrne, MD, FRCS (Plast). Al Aly, MD, FACS, The Surgical Needle, Aesthetic Surgery Journal. Volume 39, Issue Supplement_2, April 2019, Pages S73-S77, https://doi.org/10.1093/asj/sjz035
Xiong, Michael Y., and Richard G. Bennett. “Correlation of Suture Diameter With Its Ability to Cut Through an Artificial Skin Model.” Dermatologic Surgery 47.4 (2021): 480-482.
Pagnanelli. D. M., Naradzay. J. F. X., Pait. T. G., Rizzoli, H. V., & Kobrine, A. 1. (1983). The cutting-edged microvascular needle, Journal of Neurosurgery, 59(3), 510-513. Retrieved Feb. 28, 2022, from https://thejns.org/view/jourmals/j-neurosurg/59/3/article-p510.xml.
Fijalkowska M, Koziej M, Antoszewski B. Detailed head localization and incidence of skin cancers. Sci Rep 2021; 11(1):12391. DOI: 10.1038/s41598-021-91942-5.
Park S S, Hood R J. Auricular reconstruction. Otolaryngol Clin North Am 2001; 34(4):713-38, v-vi. DOI: 10.1016/s0030-6665(05)70015-3.
Aguilar E F, 3rd. Ear reconstruction. Clin Plast Surg 2004; 31(1):87-91. DOI:10.1016/s0094-1298(03)00117-2.
Kobus K, Szczyt M, Latkowski I, Wojcicki P. Reconstruction of the auricle. Br J Plast Surg 2002; 55(8):645-51. DOI: 10.1054/bjps.2002.3966.
Elsahy N I. Reconstruction of the ear after skin and cartilage loss. Clin Plast Surg 2002; 29(2):201-12, vi. DOI: 10.1016/s0094-1298(01)00004-9.
Radonich M A, Zaher M, Bisaccia E, Scarborough D. Auricular reconstruction of helical rim defects: wedge resection revisited. Dermatol Surg 2002; 28(1):62-5. DOI:10.1046/j.1524-4725.2002.01051.x.
Jackson I T. Nose Reconstruction. Local Flaps in Head and Neck Reconstruction: C. V. Mosby Company; 1985:87-188.
Pacer E, Griffin D W, Anderson A B, Tintle S M, Potter B K. Suture and Needle Characteristics in Orthopaedic Surgery. JBJS Rev 2020; 8(7):e19 00133. DOI:10.2106/JBJS.RVW.19.00133.
Dujovny M, Nossovsky N, Diaz F G, Ausman J I, Berman K S. Effects of needle shape on the integrity of the vascular endothelium. Acta Neurochir (Wien) 1985; 77(1-2):62-7. DOI:10.1007/BF01402308.
The ears. Kantor J (Ed.), (2017). Atlas of Suturing Techniques: Approaches to Surgical Wound, Laceration, and Cosmetic Repair. McGraw Hill. https://accessemergencymedicine.mhmedical.com/content.aspx?bookid=2076&sectionid=15645 5857.
Apkarian R P, Burns J T, Acland R D. Scanning electron microscopic study of disturbances in arterial walls following microsurgical needle perforations. Scan Electron Microsc 1982(Pt 2):781-7. (https://www.ncbi.nlm.nih.gov/pubmed/7167770).
Riedler K L, Shokrani A, Markarian A, Fisher L M, Pepper J P. Age-related histologic and biochemical changes in auricular and septal cartilage. Laryngoscope 2017; 127(11):E399-E407. DOI: 10.1002/lary.26807.
Bank R A, Bayliss M T, Lafeber F P, Maroudas A, Tekoppele J M. Ageing and zonal variation in post-translational modification of collagen in normal human articular cartilage. The age-related increase in non-enzymatic glycation affects biomechanical properties of cartilage. Biochem J 1998; 330 (Pt 1)(Pt 1):345-51. DOI: 10.1042/bj3300345.
Mitsou K, Papazoglou L G, Savvas I, Tzimtzimis E. Investigation of leakage holes created by four needle types used for closure of canine enterotomies. Open Vet J 2018; 8(4):411-414.
Preuss S, Eriksson E. Plastic surgery techniques. In: Achauer B M, Eriksson E, Guyuron B, Coleman III JJ, Russell R C, Vander Kolk C A, editors. Plastic Surgery Indications, Operations, and Outcomes. Vol 1. Maryland Heights: Mosby; 2001 p. 151.
Firestone D E, Lauder A J. Chemistry and mechanics of commonly used sutures and needles. J Hand Surg Am 2010; 35(3):486-8; quiz 488. DOI: 10.1016/j.jhsa.2009.10.036.
Rose J T F. Sutures and Needles. (https://www.ncbi.nlm.nih.gov/books/NBK539891/).
Robinson; JK, Hanke; CW, Siegel; DM, Fratila A. Surgery of the Skin Procedural Dermatology. Third ed. London: Elsevier Saunders, 2015.
Edlich R F, Thacker J G, McGregor W, Rodeheaver G T. Past, present, and future for surgical needles and needle holders. Am J Surg 1993; 166(5):522-32. DOI: 10.1016/s0002-9610(05)81147-x.
White J H, Stephens G M. 6/0 sutures for eyelid skin. Ophthalmic Surg 1981; 12(3):195-6. (In eng).
Pensalfini M, Meneghello S, Lintas V, Bircher K, et al. The suture retention test, revisited and revised. J Mech Behav Biomed Mater 2018; 77:711-717. DOI: 10.1016/j.jmbbm.2017.08.021.
Leslie L F, Woods J A, Thacker J G, Morgan R F, et al. Needle puncture resistance of surgical gloves, finger guards, and glove liners. J Biomed Mater Res 1996; 33(1):41-6. (In eng). DOI: 10.1002/(sici)1097-4636(199621)33:1<41::Aid-jbm7>3.0.Co; 2-m.
Longa E Z, Weinstein P R, Chater G. Scanning electron microscopy studies of needle and suture damage in rat carotid and femoral arteries. Microsurgery 1984; 5(4):169-74. (In eng). DOI: 10.1002/micr.1920050402.
Kim Y-S, Lee K-W, Kim J-S, et al. Regional thickness of facial skin and superficial fat: Application to the minimally invasive procedures. Clinical Anatomy 2019; 32(8):1008-1018. DOI: https://doi.org/10.1002/ca.23331.

What is claimed is:

1. A method of cutaneous suturing, the method comprising:
providing a suture needle comprising:
a distal end comprising a point operable to pierce the epidermis;
a proximal end attached to a suture thread;
a tapered portion between the distal and proximal end having a substantially round cross-section, wherein the suture needle does not comprise an axial cutting edge between the distal and proximal end;
piercing an epidermal layer of a patient with the point of the distal end to form an opening in the epidermal layer;
passing the suture needle through the opening; and
pulling the suture thread through the opening using the suture needle.

2. The method of claim 1 wherein the suture needle has a largest cross-sectional diameter between about 0.10 and about 0.97 mm.

3. The method of claim 1, wherein the proximal end of the suture needle is swaged to the suture thread.

4. The method of claim 1, wherein the suture thread is attached to the proximal end of the suture needle by an eye in the proximal end of the suture needle.

5. The method of claim 1, wherein the suture needle is substantially straight along its axial length.

6. The method of claim 1, wherein the suture needle is curved along its axial length.

7. The method of claim 6, wherein the suture needle is curved between about a ⅜ circle and about a ½ circle.

8. The method of claim 1, wherein the suture needle comprises a flattened or textured surface portion along its axial length between the proximal and distal ends, said flattened or textured surface portion adapted to provide grip for a suturing tool.

9. The method of claim 1, further comprising gripping the suture needle with a suturing tool at the flattened or textured surface portion during the piercing step.

10. The method of claim 1, wherein the suture thread is absorbable.

11. The method of claim 10, wherein the suture thread comprises gut, polydioxanone, poliglecaprone, or polyglactin.

12. The method of claim 1, wherein the suture thread is non-absorbable.

13. The method of claim 12, wherein the suture thread comprises one or more of polypropylene, nylon, silk, or polyester.

14. The method of claim 13, wherein the suture thread comprises polypropylene.

15. The method of claim 14, wherein the suture thread is only attached to only a single suture needle.

16. The method of claim 1, wherein the epidermal layer of the patient is located on the patient's face.

17. The method of claim 16, wherein the epidermal layer of the patient is located on the patient's eyelid.

18. The method of claim 17, wherein the suture thread is a 5-0 size or larger.

19. The method of claim 1, wherein the epidermal layer of the patient is located on the patient's abdomen.

20. The method of claim 1, wherein the epidermal layer of the patient is located on the patient's back.
